(19) United States
(12) Patent Application Publication
(10) Pub. No.: US 2023/0340403 A1
(43) Pub. Date: Oct. 26, 2023

(54) CELL-SUPPORTING BODY, MANUFACTURING METHOD THEREFOR, CELL CULTURING METHOD, AND CELL STRUCTURE

(71) Applicant: Konica Minolta, Inc., Tokyo (JP)

(72) Inventors: YASUHIKO TABATA, Uji-shi, Kyoto (JP); YUKI MURATA, Kyoto-shi, Kyoto (JP); TEMMEI ITO, Sagamihara-shi, Kanagawa (JP); AKIHIRO MAEZAWA, Hino-shi, Tokyo (JP)

(21) Appl. No.: 18/026,482

(22) PCT Filed: Sep. 17, 2020
(86) PCT No.: PCT/JP2020/035197

§ 371 Date: Mar. 15, 2023

(51) Int. Cl. C12N 5/00

(52) U.S. Cl. CPC C12N 5/0068; C12N 2513/00; C12N 2533/54

(57) ABSTRACT

The present invention relates to a cell-supporting body, including: a base material including a biocompatible substance; and a gelatin particle retained on the base material. The cell-supporting body can be manufactured by a method of retaining a gelatin particle on a base material including a biocompatible substance. By seeding a cell on the cell-supporting body and culturing the cell, a cell structure in which a reagent or an agent is uniformly incorporated into more cells can be obtained.

### TECHNICAL FIELD

The present invention relates to a cell-supporting body, a manufacturing method thereof, a cell culturing method, and a cell structure.

### BACKGROUND ART

Techniques for introducing a reagent, an agent or the like into cultured cells are known. For example, Patent Literature 1 describes that a probe solution is added to a culture solution to introduce probes into cells so that the state of the cells can be detected.

A supporting body for culturing cells for transplantation or retaining and storing cells (hereinafter, also simply referred to as a “cell-supporting body”) is known. For example, Patent Literature 2 describes a biodegradable base material including a biodegradable nonwoven fabric sewn by a biodegradable filament, and a biodegradable film-like material superimposed on the biodegradable nonwoven fabric. Patent Literature 2 describes that when the biodegradable base material is used as a cell-supporting body, cells easily enter the inside of the biodegradable base material, and the biodegradable base material can be embedded for a long period of time without causing a foreign substance reaction in a living body.

### CITATION LIST

### Patent Literature

Patent Literature 1: JP 2016-077159 A
Patent Literature 2: JP 2004-148014 A

### SUMMARY OF INVENTION

### Technical Problem

As described in Patent Literature 1, when a reagent, an agent or the like is introduced into cells, there is a problem that the reagent, the agent or the like is ununiformly incorporated into cells. In particular, when colonies of cells are grown in a three-dimensional direction, ununiformity is likely to occur in the incorporation of a reagent, an agent or the like, and cells that have incorporated these and cells that have not incorporated these are produced, or cells that have incorporated a large amount of these and cells that have incorporated only a small amount of these are produced.

The present invention has been made based on the above findings, and an object of the present invention is to provide a cell-supporting body, a manufacturing method thereof, a cell culturing method using the cell-supporting body, and a cell structure manufactured using the cell-supporting body which are capable of more uniformly introducing a reagent, an agent or the like into a cell.

### Solution to Problem

The problems are solved by a cell-supporting body, including: a base material including a biocompatible substance; and a gelatin particle retained on the base material.

The problems are also solved by a cell culturing method, including the steps of: preparing the cell-supporting body; and seeding a cell on the cell-supporting body.

The problems are also solved by a cell structure including the cell-supporting body and a cell retained on the cell-supporting body.

### Advantageous Effects of Invention

According to the present invention, a cell-supporting body, a manufacturing method thereof, a cell culturing method using the cell-supporting body, and a cell structure manufactured using the cell-supporting body which are capable of more uniformly introducing a reagent, an agent or the like into a cell are provided.

### BRIEF DESCRIPTION OF DRAWINGS

FIG. 1 is a fluorescence image observed in Experiment 1.

FIG. 2 is a fluorescence image observed in Experiment 2.

FIG. 3A is a fluorescence image observed from a cell-supporting body in which a gelatin nonwoven fabric 1 is used in Experiment 3, FIG. 3B is a fluorescence image observed from a cell-supporting body in which a gelatin nonwoven fabric 2 is used in Experiment 3, FIG. 3C is a fluorescence image observed from a cell-supporting body in which a polypropylene nonwoven fabric coated with gelatin is used in Experiment 3, and FIG. 3D is a fluorescence image observed from a cell-supporting body in which a polypropylene nonwoven fabric not coated with gelatin is used in Experiment 3.

### DESCRIPTION OF EMBODIMENTS

Hereinafter, embodiments of the present invention will be described in detail with reference to the drawings. The present invention is not limited to the following embodiments.

### Cell-Supporting Body

The cell-supporting body according to one embodiment of the present invention includes a base material including a biocompatible substance and a gelatin particle retained on the base material.

### (Base Material)

The base material can be a base material on which cells can be cultured and retained, and can be a base material having a two-dimensional shape for growing cells into planar colonies, or can be a base material having a three-dimensional shape for growing cells into three-dimensional colonies.

Examples of the base material having a two-dimensional shape include a cell culture plate, a culture dish, and a Petri dish. The base material having a two-dimensional shape can have a shape such as a bottle shape, a tube shape, a bag shape, a microchannel shape, and a multi-well plate shape.

Examples of the base material having a three-dimensional shape include an assembly of fibers such as a nonwoven fabric, a woven fabric, and a net, and a porous base material such as a membrane filter and a mesh sheet.

The material of these base materials is not particularly limited, and can be any material such as metal, resin, glass, and ceramic.

Examples of the metal include titanium, nickel, platinum, gold, tungsten, iron, and alloys thereof.

Examples of the resin include synthetic resins such as polyurethane, polyolefins such as polyethylene and polypropylene, polycarbonate, polylactic acid, polyglycolic acid, poly(ε-caprolactone), polyvinyl alcohol, and a copolymer thereof, polyethylene glycol, polyimide, acrylic resin, polyester, polyvinylidene fluoride, polyvinyl alcohol, polyvinyl chloride, polyvinyl acetate, polyvinyl pyrrolidone, polystyrene, and a copolymer thereof; natural resins such as cellulose; elastomers such as styrene-butadiene copolymers, polyisoprene, isobutylene-isoprene copolymers (butyl rubber), halogenated butyl rubbers, butadiene-styrene-acrylonitrile copolymers, silicon polymers, and fluorosilicone polymers; and bio-derived materials such as polyhydroxybutyric acid, polyhydroxyvaleric acid, proteins, sugars, and glycoproteins (such as fibronectin).

The base material can be a scaffold material that mimics an extracellular matrix for growing cells into three-dimensional colonies to form spheroids. Examples of the scaffold material include gel-like bodies of biocompatible substances described later including gelatin, collagen, and hyaluronic acid.

### (Biocompatible Substance)

The base material includes a biocompatible substance on a surface thereof.

The form of the base material is not particularly limited as long as the base material includes a biocompatible substance on a surface, and a base material having the above shapes can be formed of a biocompatible substance, or a biocompatible substance can be applied to the surface of a base material formed into a predetermined shape.

When the base material is a porous base material or the like having a three-dimensional shape, the biocompatible substance is not necessarily applied to even the inner surface of the base material, but only needs to be applied to at least the outer surface of the base material on which cells are seeded, or can be applied only to the outer surface of the base material on which cells are seeded.

The biocompatible substance can be a bio-derived polymer or a biodegradable synthetic polymer.

Examples of the bio-derived polymer include biodegradable polyesters such as polyhydroxybutyric acid and polyhydroxyvaleric acid, polysaccharides such as glycosaminoglycans (such as hyaluronic acid), starch, cellulose or derivatives thereof (such as carboxymethylcellulose), alginic acid, chitin, and chitosan, polyamino acids such as collagen, elastin, gelatin, and laminin, glycoproteins such as fibronectin, and complexes thereof. Among these, collagen, gelatin, fibronectin, laminin, and polysaccharides are preferable.

Examples of the biodegradable synthetic polymer include polylactic acid, polyglycolic acid, poly(ε-caprolactone), polyvinyl alcohol, and a copolymer thereof, polyethylene glycol, polyhydroxybutyric acid, polyhydroxyvaleric acid, and biodegradable polyesters. Among these, polylactic acid, polyglycolic acid, poly(ε-caprolactone), polyvinyl alcohol, and a copolymer thereof are preferable.

According to findings by the present inventors, among these biocompatible substances, by a polymer material having a higher water content, the introduction rate of the gelatin particle into a cell can be easily increased and controlled. From the above viewpoint, the biocompatible substance is preferably collagen, gelatin, fibronectin, polyvinyl alcohol, a copolymer thereof, chitin, and chitosan, and more preferably collagen, gelatin, fibronectin, and polyvinyl alcohol.

Among these biocompatible substances, polyamino acids are preferable, and gelatin is more preferable because the affinity for the gelatin particle is high and the introduction rate of the gelatin particle into a cell can be easily controlled.

The gelatin can be any known gelatin obtained by modifying collagen derived from bovine bone, cow skin, pig skin, pig tendon, fish scales, fish meat and the like.

The gelatin can be crosslinked. The crosslinking can be crosslinking with a crosslinker, or can be self-crosslinking performed without using a crosslinker.

The crosslinker can be, for example, a compound having multiple functional groups that form a chemical bond with a hydroxyl group, a carboxyl group, an amino group, a thiol group, an imidazole group or the like. Examples of such crosslinker include glutaraldehyde, water soluble carbodiimides including 1-ethyl-3-(3-dimethylaminopropyl)carbodiimide hydrochloride (EDC) and 1-cyclohexyl-3-(2-morpholinoethyl)carbodiimide-metho-p-toluenesulfonate (CMC), compounds having two or more epoxy groups including ethylene glycol diglycidyl ether, polyethylene glycol diglycidyl ether, polyglycerol polyglycidyl ether, and glycerol polyglycidyl ether, and propylene oxide. Among these, glutaraldehyde and EDC are preferable, and glutaraldehyde is more preferable from the viewpoint of further increasing the reactivity.

Examples of the self-crosslinking include crosslinking by application of heat or irradiation with an electron beam or an ultraviolet ray.

In the present embodiment, the introduction rate of the gelatin particle into a cell to be seeded and grown can be controlled by the type of the biocompatible substance. For example, when the introduction rate of the gelatin particle into a cell is desired to be increased, it is preferable to use a biocompatible substance closer to gelatin, or to make the degree of crosslinking of gelatin as the biocompatible substance closer to the degree of crosslinking of gelatin in the gelatin particle. Alternatively, when the introduction rate of the gelatin particle into a cell is desired to be suppressed to some extent, it is preferable to use a biocompatible substance more different to gelatin, or to make the degree of crosslinking of gelatin as the biocompatible substance more different to the degree of crosslinking of gelatin in the gelatin particle. Thus, it is desirable to select the type of the biocompatible substance according to the introduction rate of the gelatin particle to be achieved.

The difference in the degree of crosslinking of the gelatin can be estimated from a difference in signal intensity of any energy band in a loss spectrum detected by transmission electron microscope observation (TEM-EELS measurement) in combination with electron energy-loss spectroscopy, a peak intensity ratio between COOH and CONH (peak intensity of COOH/peak intensity of CONH) in a spectrum in which wave numbers are plotted on the horizontal axis and an absorbances are plotted on the vertical axis, which is obtained by measurement with a Fourier transform type infrared spectrophotometer (FT-IR), or the like.

By controlling the introduction rate of the gelatin particle, the introduction rate of a reagent or an agent carried by the gelatin particle into a cell can also be controlled. Depending on the type of the reagent or the agent, the amount to be introduced into a cell varies. Therefore, by controlling the introduction rate of the gelatin particle into a cell by the biocompatible substance, and also controlling the introduction rate of the reagent or the agent into the cell, the amount of the reagent or the agent introduced into the cell can be easily controlled.

### (Gelatin Particle)

The base material retain a gelatin particle.

The gelatin particle is in contact with the biocompatible substance of the base material and is immobilized on the base material.

At this time, the gelatin particle is immobilized and retained at a position in contact with the biocompatible substance included in the base material. For example, when the biocompatible substance is applied to the surface of the base material, the gelatin particle is applied and immobilized in a region of the base material to which the biocompatible substance is applied.

When the base material is a porous base material or the like having a three-dimensional shape, the gelatin particle is not necessarily applied to even the inner surface of the base material, but only needs to be applied to at least the outer surface of the base material on which cells are seeded, or can be applied only to the outer surface of the base material on which cells are seeded.

The gelatin particle can be a nanoparticle composed of any known gelatin same as those described for the biocompatible substance. Gelatin has been used for food and medical purposes for a long time, and is less harmful to the human body even when ingested into the body. Because gelatin is dispersed and lost in a living body, advantageously there is no need to remove the gelatin from the living body.

The weight average molecular weight of the gelatin that constitutes the gelatin particle is preferably 1000 or more and 100,000 or less. The weight average molecular weight can be, for example, a value measured according to PAGI Method 10th Edition (2006).

The gelatin that constitutes the gelatin particle can be crosslinked. The crosslinking can be crosslinking with the above crosslinkers, or can be self-crosslinking performed without using a crosslinker.

From the viewpoint of easily controlling the ease of incorporation into a cell, the gelatin particle is preferably cationized, for example, by introduction of a primary amino group, a secondary amino group, a tertiary amino group, or a quaternary ammonium group.

Cationization of the gelatin particle can be performed by a known method in which a functional group that is cationized under physiological conditions is introduced during manufacturing. For example, the above amino groups can be introduced into a hydroxyl group or a carboxyl group of gelatin by reacting alkyldiamines such as ethylenediamine and N,N-dimethyl-1,3-diaminopropane, trimethylammonium acetohydrazide, spermine, spermidine, and diethylamide chloride using a condensing agent such as 1-ethyl-3-(3-dimethylaminopropyl)carbodiimide hydrochloride, cyanuric chloride, N,N′ -carbodiimidazole, cyanogen bromide, diepoxy compounds, tosyl chloride, dianhydride compounds such as diethyltriamine-N,N,N′,N″,N″ -pentanoic acid dianhydride, and tricyl chloride.

The average particle size of the gelatin particle is preferably 100 nm or more and 1000 nm or less. Although the gelatin particle carries a probe, the gelatin particle do not substantially have a probe in a surface layer thereof. Thus, even when the average particle size is 1000 nm or less, the gelatin particle is easily incorporated into a cell by the cell’ own activity. To allow many gelatin particles to be incorporated into cells in a shorter time, the average particle size of the gelatin particle is more preferably 800 nm or less. On the other hand, a gelatin particle having an average particle size of 100 nm or more can easily carry a probe in the particle, and the capacity of the probe can be increased. The larger the average particle size of the gelatin particle is within the range of 1000 nm or less, the more easily the gelatin particle is incorporated into a cell by the cell’ own activity. From the above viewpoint, the average particle size of the gelatin particle is preferably 200 nm or more, and more preferably 300 nm or more.

The average particle size of the gelatin particle can be an apparent particle size of the gelatin particle measured by dynamic light scattering. Alternatively, the average particle size of the gelatin particle can be a value obtained by averaging the major axis and the minor axis. The minor axis and major axis of the gelatin particle can be values obtained by analyzing an image obtained by imaging a dried gelatin particle after being left to stand in the atmosphere at 80° C. for 24 hours with a scanning electron microscope (SEM). Because gelatin particles are usually an aggregate composed of multiple gelatin particles, each of the major axis, minor axis, and particle size of gelatin particles can be a value obtained by averaging the major axis, minor axis, and particle size of multiple gelatin particles (for example, 20 gelatin particles) selected at random from the aggregate. When there is a difference between the average particle sizes measured by these methods, the average particle size obtained by measurement by dynamic light scattering can be adopted.

The gelatin particle carries a reagent or an agent.

The gelatin particle carrying a probe means that the probe is immobilized on the surface of the gelatin particle or incorporated inside the gelatin particle.

In the gelatin particle, the amount of probes inside the gelatin particle is preferably larger than the amount of probes in the surface layer. By reducing the amount of probes in the surface layer of the gelatin particle, the amount of probes exposed on the surface of the gelatin particle can be reduced. As a result, the gelatin particle becomes less likely to be recognized as a foreign substance by cells, and can be easily incorporated into cells by an activity such as endocytosis. The surface layer means a region up to a depth of 1% with respect to the average particle size of the gelatin particle.

The reagent can be a probe used for applications such as examination of biological activity and the like, measurement of a substance in a living body, and quantification of a substance in a living body, a contrast medium for detecting the presence of cells, and the like. The detection target of the probe is not particularly limited, and can be a protein, a sugar, or a nucleic acid such as DNA and mRNA, or can be a physiological state such as temperature and pH in a cell.

The probe can be, for example, a compound having a site that directly or indirectly binds to a substance to be detected and a site that emits a detectable signal. For example, the probe can be a probe capable of specifically binding to mRNA to be detected by a nucleic acid having a sequence complementary to at least a part of the nucleic acid sequence of the mRNA to be detected, or can be a probe capable of specifically binding to a protein to be detected by an antibody. The probe can be a probe that contains a phosphor and emits fluorescence as a signal, or can be a probe that emits another signal by chemiluminescence or the like.

The type of the phosphor is not particularly limited, and can be a fluorescent dye or a semiconductor nanoparticle.

Examples of the fluorescent dye include a rhodamine dye molecule, a squarylium dye molecule, a fluorescein dye molecule, a coumarin dye molecule, an acridine dye molecule, a pyrene dye molecule, an erythrosin dye molecule, an eosin dye molecule, a cyanine dye molecule, an aromatic ring dye molecule, an oxazine dye molecule, a carbopyronine dye molecule, and a pyrromethene dye molecule.

Examples of the semiconductor that constitutes the semiconductor nanoparticle include a II-VI compound semiconductor, a III-V compound semiconductor, and a IV semiconductor. Specific examples of the semiconductor that constitutes the semiconductor nanoparticle include CdSe, CdS, CdTe, ZnSe, ZnS, ZnTe, InP, InN, InAs, InGaP, GaP, GaAs, Si, and Ge.

The probe capable of specifically binding to the mRNA can be a known probe such as a molecular beacon, a Taqman probe, a cycling probe, and an INAF probe, but a molecular beacon is preferable because a general-purpose fluorescent dye can be used and detection for various cell types is easy.

A molecular beacon is a nucleic acid derivative having a stem-loop structure, in which a fluorescent dye is bound to one end of the 5 ′ end and the 3 ′ end, and a quenching dye is bound to the other end. In the molecular beacon, the fluorescent dye and the quenching dye are close to each other, and thus the fluorescence emitted from the fluorescent dye is quenched in a state where the stem-loop structure is formed. When the molecular beacon is close to a target sequence, the loop structure is opened, and the molecular beacon binds to mRNA to be detected. As a result, the fluorescent dye and the quenching dye are separated from each other, and fluorescence emission is detected.

The combination of the fluorescent dye and the quenching dye is not particularly limited, and can be appropriately selected from the fluorescent dyes described above. The quenching dye can be a molecule that performs quenching by any of fluorescence resonance energy transfer (FRET), contact quenching, and collisional quenching.

The probe capable of specifically binding to the protein to be detected by the antibody is preferably a Phosphor Integrated Dot (PID). The PID is a nano-sized particle having an organic or inorganic particle as a main material and containing multiple phosphors. The PID specifically and directly or indirectly binds to the protein to be detected by the antibody to label the protein to be detected. The multiple phosphors can be present inside the particle or on the surface of the particle. The phosphor integrated particle can emit fluorescence having an intensity sufficient to indicate the target substance molecule as a bright spot one by one.

Examples of the organic substance as a main material include thermosetting resins such as a melamine resin, a urea resin, an aniline resin, a guanamine resin, a phenol resin, a xylene resin, and a furan resin; thermoplastic resins such as a styrene resin, an acrylic resin, an acrylonitrile resin, an AS resin (acrylonitrile-styrene copolymer), and an ASA resin (acrylonitrile-styrene-methyl acrylate copolymer); other resins such as polylactic acid; and polysaccharides. Examples of the inorganic substance as a main material include silica and glass. The main material and the fluorescent substance are preferably those having substituents or sites having charges opposite to each other, and having electrostatic interaction.

The average particle size of the phosphor integrated particle is not particularly limited, but is preferably 10 nm or more and 500 nm or less, and more preferably 50 nm or more and 200 nm or less in consideration of ease of detection as a bright spot.

The particle size of the phosphor integrated particle can be measured by measuring the projected area of the phosphor integrated particle using a scanning electron microscope (SEM) and converting the projected area into an equivalent circle diameter. The average particle size and the coefficient of variation of a group consisting of multiple phosphor integrated particles are calculated using the particle size (equivalent circle diameter) calculated for a sufficient number (for example, 1000) of phosphor integrated particles.

The agent can be any agent that can be carried by the gelatin particle. Examples of such an agent include proteins having pharmaceutical activity, nucleic acids used in pharmaceutical applications including plasmids, aptamers, antisense nucleic acids, ribozymes, tRNAs, snRNAs, siRNAs, shRNAs, ncRNAs, and condensed DNAs, and antigens used in pharmaceutical applications.

Examples of the proteins having pharmaceutical activity include steroids, non-steroidal anti-inflammatory drugs (NSAID), vitamin A (retinoid), vitamin D3 and vitamin D3 analogs, antibiotics, antiviral agents, and antibacterial agents.

The agent can be a water-soluble agent or a water-insoluble agent. Examples of the water-insoluble agent include immunosuppressive agents such as cyclosporines including cyclosporine, immunoactive agents such as rapamycin, anticancer agents such as paclitaxel, antiviral or antibacterial agents, anti-neoplastic tissue agents, analgesics and anti-inflammatory agents, antibiotics, anti-epileptic agents, anxiolytic agents, anti-anesthetics, antagonists, neuron blocking agents, anti-cholinergic agents, anti-arrhythmic agents, anti-hypertensive agents, hormonal agents, and nutritional agents.

As these water-insoluble agents, rapamycin, paclitaxel, docetaxel, and everolimus are preferable. Rapamycin, paclitaxel, docetaxel, and everolimus each include analogs and derivatives thereof as long as they have similar drug efficacy. For example, paclitaxel and docetaxel are in an analog relationship, and rapamycin and everolimus are in a derivative relationship. Among these, paclitaxel is more preferable.

The reagent or the agent can be pre-coated on the base material in addition to being carried on the gelatin particle. However, when the reagent or the agent is coated on the base material and incorporated into cells, the ease of incorporation into cells is likely to vary. Therefore, the amount carried on the gelatin particle is preferably larger than the amount coated on the base material (mass standard), and more preferably coating on the base material is not performed.

### Cell Culturing Method

The cell culturing method according to another embodiment of the present invention is a cell culturing method in which the above cell-supporting body is used.

The cell culturing according to the present embodiment can be performed in the same manner as known cell culturing methods except for using the above cell-supporting body as a cell-supporting body.

Specifically, the cell culturing method according to the present embodiment can include the steps of: preparing the above cell-supporting body; and seeding a cell on the above cell-supporting body.

For preparation of the cell-supporting body, a cell-supporting body that has already been prepared can be prepared, or a cell-supporting body can be produced.

The cell-supporting body can be produced by including the steps of: preparing a base material including a biocompatible substance; and retaining the gelatin particle on the base material.

The step of preparing a base material including a biocompatible substance is a step of preparing a base material having the above shape and composed of the above material. When the base material is a formed body formed from a biocompatible substance, the formed body may be prepared. Alternatively, a biocompatible substance can be applied to a known base material for coating and the like. The biocompatible substance can be applied by, for example, a method in which a solution containing the biocompatible substance is applied to the surface of the base material, and then the solution is dried. When the base material is a porous base material or the like having a three-dimensional shape, the biocompatible substance is not necessarily applied to even the inner surface of the base material, but only needs to be applied to at least the outer surface of the base material on which cells are seeded, or can be applied only to the outer surface of the base material on which cells are seeded.

In the step of retaining the gelatin particle, a solution containing the gelatin particle can be applied to the base material including the biocompatible substance. At this time, by heating the gelatin particle to a temperature at which the gelatin particle is solated (about 35° C. to 45° C.) and allowing the gelatin particle to stand for about 1 hour, the gelatin particle can be immobilized on the base material due to adhesiveness exhibited by the solated gelatin particle, and the gelatin particle can be retained on the base material. The solution can then be dried.

At this time, the gelatin particle is immobilized and retained at a position in contact with the biocompatible substance included in the base material. For example, when the biocompatible substance is applied to the surface of the base material, the gelatin particle is applied and immobilized in a region of the base material to which the biocompatible substance is applied. When the base material is a porous base material or the like having a three-dimensional shape, the gelatin particle is not necessarily applied and immobilized to even the inner surface of the base material, but only needs to be applied and immobilized to at least the outer surface of the base material on which cells are seeded, or can be applied and immobilized only to the outer surface of the base material on which cells are seeded.

The gelatin particle carries a reagent or an agent. The carrying of the reagent or the agent by the gelatin particle can be achieved by a known method in which the gelatin particle is mixed with the reagent or the agent, or the reagent or the agent is added to a solution for preparing the gelatin particle.

Also at this time, when the base material is a porous base material or the like having a three-dimensional shape, the gelatin particle is not necessarily applied and immobilized to even the inner surface of the base material, but only needs to be immobilized and retained on at least the outer surface of the base material on which cells are seeded.

The cells can be seeded by a normal method.

The cells to be seeded can be cells desired to be cultured and stored as necessary. Examples of the cells include known cells including cells derived from biological samples or specimens extirpated from various organs including bone marrow, heart, lung, liver, kidney, pancreas, spleen, intestinal tract, small intestine, heart valve, skin, blood vessel, cornea, eyeball, dura mater, bone, trachea, and ossicle, commercially available established cell lines, stem cells including skin stem cells, epidermal keratinized stem cells, retinal stem cells, retinal epithelial stem cells, cartilage stem cells, hair follicle stem cells, muscle stem cells, bone progenitor cells, adipose progenitor cells, hematopoietic stem cells, neural stem cells, hepatic stem cells, pancreatic stem cells, ectodermal stem cells, mesodermal stem cells, endodermal stem cells, mesenchymal stem cells, ES cells, and iPS cells, and cells differentiated from these stem cells. The cells to be seeded can also be cells of organisms other than animals, such as plants, fungi, protists, and bacteria.

At this time, a known medium can be added to the cell retainer to promote the growth of cells.

The seeded cells proliferate using the cell retainer as a scaffold and form colonies while incorporating the gelatin particle retained by the cell retainer by endocytosis. Then, the reagent or the agent carried by the gelatin particle is slowly released from the gelatin particle incorporated into the cells. In this way, colonies are formed by cells containing the reagent or the agent.

Conventionally, an attempt has been made to incorporate a reagent or an agent into cells from a solution containing the reagent or the agent, or an attempt has been made to introduce a reagent or an agent into cells by applying a dispersion containing gelatin particles carrying the reagent or the agent to a medium to incorporate the gelatin particles. According to the findings of the present inventors, when the gelatin particle retained by the cell retainer is incorporated into cells, a reagent or an agent can be uniformly introduced into more cells as compared with these methods.

When the base material is a porous base material or the like having a three-dimensional shape, cells can be seeded in a region retaining the gelatin particle on the surface of the cell retainer. The seeded cells incorporate the gelatin particle retained by the cell retainer by endocytosis. Then, when the cells that have incorporated the gelatin particle proliferate while dividing and enter the inside of the base material, presumably, the reagent or the agent carried by the gelatin particle is also carried by each of the divided cells to form colonies by the cells containing the reagent or the agent.

### Cell Structure

The cell structure according to another embodiment of the present invention is a cell structure including the above cell-supporting body.

The cell structure includes a base material including the biocompatible substance described above and multiple cells instructed by the cell-supporting body. The gelatin particle retained by the base material and the reagent or the agent carried by the gelatin particle are incorporated into the cells.

In the cell culture body, the gelatin particle, or the reagent or the agent is uniformly incorporated into more cells. For example,

The cell culture body can be used for transplantation into a living body, storage of cells and the like. In such a case, because the reagent or the agent carried by the gelatin particle is uniformly incorporated into more cells, detection of a state of, for example, differentiation of cells, detection of life and death of cells and the like can be performed with higher sensitivity.

### EXAMPLES

Hereinafter, specific Examples of the present invention will be described together with Comparative Examples, but the present invention is not limited thereto.

### 1. Probe

The following probe was used.

GAPDH-MB: A probe in which the 5′ end of a base sequence containing a sequence complementary to mRNA of glutaraldehyde-3-phosphate dehydrogenase (GAPDH) is modified with AlexaFlour 488, and the 3′ end is modified with IBRQ (lowa black RQ). GAPDH-MB is a molecular beacon in which the 5′ end site and the 3′ end site are complementary sequences that constitute a stem region, and a site between them is a sequence that constitutes a loop structure.

It has been confirmed in advance that for the fluorescence intensity from the molecular beacon, fluorescence is emitted only when the molecular beacon reacted with mRNA of glutaraldehyde-3-phosphate dehydrogenase (GAPDH), which is mRNA constantly expressed in cells, and that the fluorescence intensity is increased according to each amount of mRNA.

### 2. Experiment 1

### 2-1. Preparation of Gelatin Particle

Gelatin (G-2613 P manufactured by Nitta Gelatin Inc.) was dissolved in 24 ml of a 0.1 M phosphate buffered aqueous solution (pH 5.0) at 37° C. To this solution, an appropriate amount of ethylenediamine was added. Further, an aqueous hydrochloric acid solution was added to adjust the pH of the solution to 5.0. Further, an appropriate amount of 1-ethyl-3-(3-dimethylaminopropyl)carbodiimide hydrochloride was added, and the concentration of gelatin was adjusted to 2% by mass by addition of a 0.1 M phosphate buffered aqueous solution. This solution was stirred at 37° C. for 4 hours to introduce ethylenediamine into the carboxyl group of the gelatin. Then, the reaction product was dialyzed in redistilled water for 3 days to obtain a cationized gelatin in a slurry state. Then, acetone as a phase separation inducer was added, the mixture was mixed at 50° C., and the particles precipitated in the slurry were recovered and washed with pure water to obtain cationized gelatin particles. The cationized gelatin particles are referred to as cGNS.

The apparent average particle size of cGNS was determined by dynamic light scattering at 37° C. using DLS-7000 manufactured by OTSUKA ELECTRONICS CO.,LTD, and found to be 168.0 nm. The zeta potential of cGNS was determined by electrophoretic light scattering using DLS-8000 manufactured by OTSUKA ELECTRONICS CO.,LTD, and found to be 8.41 mV.

### 2-2. Carrying of Molecular Beacon by Gelatin Particle

cGNS and GAPDH-MB were mixed at room temperature for 15 minutes, and then centrifuged and washed with water to obtain gelatin particles carrying the probe. The gelatin particles are designated as cGNS (GAPDH-MB).

### 2-3. Retaining of Gelatin Particle by Base Material

As a base material having a two-dimensional shape, a cell culture plate (96 well Edge Plate, manufactured by Thermo Fisher Scientific) was prepared. This cell culture plate was coated with gelatin (manufactured by Nitta Gelatin Inc., Type B, isoelectric point 5).

To the base material, cGNS (GAPDH-MB) was added, and the base material was allowed to stand at 37° C. for 1 hour to immobilize cGNS (GAPDH-MB), thereby a cell-supporting body was obtained.

### 2-4. Culturing of Cell on a Cell-Supporting Body

MC3T3-E1 cells, a mouse mesenchymal stem cell line, were seeded on the cell-supporting body, and the cells were cultured for one day.

### 2-5. Observation

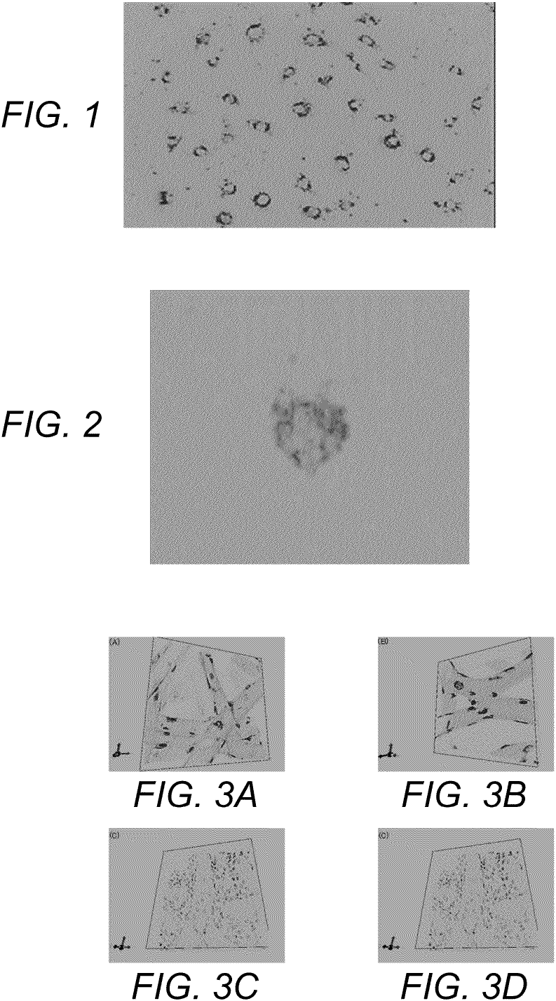

FIG. 1 is a fluorescence image observed one day after seeding. As is apparent from FIG. 1, red fluorescence was uniformly observed from the entire plate. This is presumably because GAPDH-MB was uniformly incorporated into all of cells grown on the cell-supporting body, and reacted with mRNA of GAPDH in the cells to emit red fluorescence.

### 3. Experiment 2

### 3-1. Preparation of Gelatin Particle and Carrying of Molecular Beacon by Gelatin Particle

In the same manner as in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 3-2. Retaining of Gelatin Particle by Base Material

Gelatin hydrogel particles were dispersed in PBS to prepare swollen thermally crosslinked gelatin hydrogel particles (scaffold material for spheroid formation). The particle size of the thermally crosslinked gelatin hydrogel particles was 32 to 53 mm. The thermally crosslinked gelatin hydrogel particles and gelatin particles cGNS (GAPDH-MB) were mixed and allowed to stand at 37° C. for 1 hour, and cGNS (GAPDH-MB) was immobilized on the thermally crosslinked gelatin hydrogel particles to obtain a cell-supporting body.

### 3-3. Seeding of Cell to Cell-Supporting Body

The cell-supporting body and MC3T3-E1 cells, a mouse mesenchymal stem cell line, were introduced into a polyvinyl alcohol-coated U-bottomed 96 well plate, and the cells were cultured for 3 days to form cell aggregates.

### 3-4. Observation

Three days after seeding, the cell aggregates were fixed with 4% paraformaldehyde, and a frozen section of the center plane of the cell aggregates was produced. Nuclei were stained by adding 4′,6-diamidino-2-phenylindole (DAPI) to the frozen section, and then observation was performed with a fluorescence microscope.

FIG. 2 is a fluorescence image observed at this time. As is apparent from FIG. 2, red fluorescence was uniformly observed from the entire plate. This is presumably because GAPDH-MB was uniformly incorporated into all of cells grown into cell aggregates, and reacted with mRNA of GAPDH in the cells to emit red fluorescence.

### 4. Experiment 3

### 4-1. Preparation of Gelatin Particle

In the same manner as in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 4-2. Retaining of Gelatin Particle by Base Material

A gelatin nonwoven fabric 1 that is not thermally crosslinked (Genocel manufactured by THE JAPAN WOOL TEXTILE CO., Ltd. (“Genocel” is a registered trademark of the company)) was prepared. To the base material, cGNS (GAPDH-MB) was added, and the base material was allowed to stand at 37° C. for 1 hour to immobilize cGNS (GAPDH-MB), thereby a cell-supporting body was obtained.

### 4-3. Culturing of Cell

MC3T3-E1 cells, a mouse mesenchymal stem cell line, were seeded on the cell-supporting body, and the cells were cultured for one day. As a comparative example, MC3T3-E1 cells were seeded on a 6-well plate, and the cells were cultured for one day in the presence of an OptiMEM culture solution to which cGNS (GAPDH-MB) was added.

### 4-4. Observation

One day after seeding, the cultured cells were observed with a fluorescence microscope, and the imaging fluorescence intensity of six randomly imaged fields was measured. The imaging fluorescence intensity was divided by the number of cells confirmed in each field to calculate the average fluorescence intensity per number of cells, and the standard deviation for each field was also calculated.

Table 1 shows the average fluorescence intensity per number of cells and standard deviation.

TABLE 1

| | Gelatin particle | Average fluorescence intensity per cell | Standard deviation |
|---|---|---|---|
| Example | Immobilized on base material (cell-supporting body) | 16.8 | 2.6 |
| Comparative Example | Added to culture solution | 12.1 | 5.6 |

As shown in Table 1, when the cell-supporting body was used, the average fluorescence intensity per cell was higher and the standard deviation was smaller. From this result, it was found that when a cell-supporting body including a base material including a biocompatible substance and a gelatin particle retained on the base material is used, incorporation of a molecular beacon (agent or reagent) by cells is promoted, and the molecular beacon is incorporated more uniformly.

### 5. Experiment 4

### 5-1. Preparation of Gelatin Particle

In the same manner as in Experiment 1, cationized gelatin particles cGNS were prepared. At this time, three types of cGNS (cGNS1 to cGNS3) having different average particle sizes were adjusted by changing the preparation conditions. Each of cGNS1 to cGNS3 and GAPDH-MB were mixed at room temperature for 15 minutes, and then centrifuged and washed with water to obtain gelatin particles cGNS1 (GAPDH-MB) to cGNS3 (GAPDH-MB) carrying the probe.

### 5-2. Retaining of Gelatin Particle by Base Material

A gelatin nonwoven fabric that is not thermally crosslinked (Genocel manufactured by THE JAPAN WOOL TEXTILE CO., Ltd.) was prepared. To this base material, each of cGNS1 (GAPDH-MB) to cGNS3 (GAPDH-MB) was added, and the base material and each of cGNS1 (GAPDH-MB) to cGNS3 (GAPDH-MB) were allowed to stand at 37° C. for 1 hour to immobilize cGNS1 (GAPDH-MB) to cGNS3 (GAPDH-MB), thereby cell-supporting bodies 1 to 3 were obtained.

### 5-3. Culturing of Cell

MC3T3-E1 cells, a mouse mesenchymal stem cell line, were seeded on each of the cell-supporting body 1 to the cell-supporting body 3, and the cells were cultured for one day.

### 5-4. Observation

One day after seeding, the cultured cells were observed with a fluorescence microscope, and the imaging fluorescence intensity of six randomly imaged fields was measured. The imaging fluorescence intensity was divided by the number of cells confirmed in each field to calculate the average fluorescence intensity per number of cells.

Table 2 shows the average fluorescence intensity per number of cells.

TABLE 2

| Average particle size of gelatin particle (nm) | Average fluorescence intensity per cell |
|---|---|
| 25 | 2.5 |
| 50 | 4.3 |
| 500 | 21.3 |

As shown in Table 2, it was found that the larger the average particle size of the gelatin particle, the more easily the gelatin particle and the molecular beacon (agent or reagent) carried on the gelatin particle are incorporated into cells by the cells’ own activity.

### 6. Experiment 5

### 6-1. Preparation of Gelatin Particle

In the same manner as in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 6-2. Retaining of Gelatin Particle by Base Material

As base materials having a three-dimensional shape, a gelatin nonwoven fabric 1 that is not thermally crosslinked (Genocel manufactured by THE JAPAN WOOL TEXTILE CO., Ltd.), a thermally crosslinked gelatin nonwoven fabric 2 (Genocel manufactured by THE JAPAN WOOL TEXTILE CO., Ltd., heat treatment time: 4 to 24 hours), and a polypropylene nonwoven fabric (polypropylene long fiber nonwoven fabric manufactured by TORAY INDUSTRIES, INC.) were prepared. For the polypropylene nonwoven fabric, a base material coated with gelatin and a base material not coated with gelatin were prepared.

To these base materials, cGNS (GAPDH-MB) was added, and the base materials and cGNS (GAPDH-MB) were allowed to stand at 37° C. for 1 hour to immobilize cGNS (GAPDH-MB), thereby cell-supporting bodies were obtained.

### 6-3. Seeding of Cell to Cell-Supporting Body

MC3T3-E1 cells, a mouse mesenchymal stem cell line, were seeded on the cell-supporting body, and the cells were cultured for one day.

### 6-4. Observation

One day after seeding, PBS containing CYTO 13 was added to each cell-supporting body to stain the nuclei, and the cell-supporting body and the PBS were allowed to stand at 37° C. for 1 hour. Then, each cell-supporting body was reflected on a glass bottom dish and observed with a confocal laser microscope.

FIG. 3A is a fluorescence image observed from the cell-supporting body in which gelatin nonwoven fabric 1 is used. FIG. 3B is a fluorescence image observed from the cell-supporting body in which gelatin nonwoven fabric 2 is used. FIG. 3C is a fluorescence image observed from the cell-supporting body in which a polypropylene nonwoven fabric coated with gelatin is used. FIG. 3D is a fluorescence image observed from the cell-supporting body in which a polypropylene nonwoven fabric not coated with gelatin is used.

As is apparent from FIG. 3A to FIG. 3C, red fluorescence was uniformly observed from the entire plate. This is presumably because GAPDH-MB was uniformly incorporated into all of cells grown into cell aggregates, and reacted with mRNA of GAPDH in the cells to emit red fluorescence. The fluorescence intensity was different depending on the type of the base material. From this result, it was found that the introduction rate of GAPDH-MB varies depending on the state of base materials, and it was found that the introduction rate of GAPDH-MB can be changed by changing the state of base materials.

On the other hand, as apparent from FIG. 3D, when cGNS (GAPDH-MB) was immobilized on the base material to which a biocompatible substance was not added, and cells were cultured, red fluorescence was not observed. From this result, it was found that the incorporation of cGNS (GAPDH-MB) is promoted when the base material has a biocompatible substance.

### 7. Experiment 6

### 7-1. Preparation of Gelatin Particle

In the same manner as in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 7-2. Retaining of Gelatin Particle by Base Material

As base materials, a polypropylene (PP) well plate, a polypropylene (PP) nonwoven fabric (polypropylene long fiber nonwoven fabric manufactured by TORAY INDUSTRIES, INC.), a well plate coated with polyvinyl alcohol (PVA), a well plate coated with gelatin, and a gelatin nonwoven fabric (Genocel manufactured by THE JAPAN WOOL TEXTILE CO., Ltd.) were prepared. Each of cGNS (GAPDH-MB) was added to these base materials, and the base materials and each of cGNS (GAPDH-MB) were allowed to stand at 37° C. for 1 hour to immobilize cGNS1 (GAPDH-MB), thereby cell-supporting bodies 1 to 5 were obtained.

### 7-3. Culturing of Cell

MC3T3-E1 cells, a mouse mesenchymal stem cell line, were seeded on each of the cell-supporting body 1 to the cell-supporting body 5, and the cells were cultured for one day.

### 7-4. Observation

One day after seeding, the cultured cells were observed with a fluorescence microscope, and the imaging fluorescence intensity of six randomly imaged fields was measured. The imaging fluorescence intensity was divided by the number of cells confirmed in each field to calculate the average fluorescence intensity per number of cells.

Table 3 shows the average fluorescence intensity per number of cells.

TABLE 3

| Cell-supporting body | Material of base material | Average fluorescence intensity per cell |
|---|---|---|
| 1 | PP | 0.5 |
| 2 | PP nonwoven fabric | 0.7 |
| 3 | PVA coating | 13.8 |
| 4 | Gelatin coating | 16.7 |
| 5 | Gelatin nonwoven fabric | 21.3 |

As shown in Table 3, when the base material included a biocompatible substance, the average fluorescence intensity per cell was high. From this result, it was found that the incorporation of cGNS (GAPDH-MB) is promoted when the base material has a biocompatible substance.

### 8. Experiment 7

### 8-1. Preparation of Gelatin Particle

In the same manner as in Experiment 1, cationized gelatin particles cGNS were prepared. cGNS and GAPDH-MB were mixed at room temperature for 15 minutes, and then centrifuged and washed with water to obtain gelatin particles cGNS (GAPDH-MB) carrying the probe.

### 8-2. Preparation of Base Material

A gelatin nonwoven fabric that is not thermally crosslinked (Genocel manufactured by THE JAPAN WOOL TEXTILE CO., Ltd.) was prepared. To the base material, cGNS (GAPDH-MB) was added, and the base material was allowed to stand at 37° C. for 1 hour to immobilize cGNS (GAPDH-MB), thereby a cell-supporting body was obtained.

As a comparative example, a base material obtained by applying an agent (paclitaxel) dissolved in a solvent (absolute ethanol) to a base material (gelatin nonwoven fabric (Genocel manufactured by THE JAPAN WOOL TEXTILE CO., Ltd.)), and drying them to coat the base material with the agent were prepared.

### 8-3. Culturing of Cell

SK-BR-3 cells, a human breast cancer cell line, were seeded on each of the prepared cell-supporting body and the base material coated with the agent, and the cells were cultured for 24 to 48 hours.

### 8-4. Observation

The cell aggregates were fixed with 4% paraformaldehyde, and a frozen section of the center plane of the cell aggregates was produced. Nuclei were stained by adding 4′,6-diamidino-2-phenylindole (DAPI) to the frozen section, and then observed with a fluorescence microscope, and the imaging fluorescence intensity of six randomly imaged fields was measured. The imaging fluorescence intensity was divided by the number of cells confirmed in each field to calculate the average fluorescence intensity per number of cells, and the standard deviation for each field was also calculated.

Table 4 shows the average fluorescence intensity per number of cells and standard deviation.

TABLE 4

| | Agent | Average fluorescence intensity per cell | Standard deviation |
|---|---|---|---|
| Example | Immobilization of gelatin particle carrying agent on base material (cell-supporting body) | 18.9 | 1.8 |
| Comparative Example | Coating on base material | 30.8 | 5.8 |

As shown in Table 4, when the cell-supporting body was used, the standard deviation was smaller. On the other hand, it was found that when the base material was coated with only the agent, though it is seen from the average fluorescence intensity per cell that the agent was sufficiently incorporated into cells, the standard deviation was large, and the incorporation of the agent into cells varied. From this result, it was found that when a cell-supporting body including a base material including a biocompatible substance and a gelatin particle retained on the base material is used, an agent or a reagent is incorporated more uniformly by cells.

### INDUSTRIAL APPLICABILITY

According to the present invention, a cell structure in which a reagent or an agent is uniformly incorporated into more cells can be obtained. This cell structure can be suitably used for transplantation and storage of cells.

1. A cell-supporting body, comprising:
a base material including a biocompatible substance; and
a gelatin particle retained on the base material.

2. The cell-supporting body according to claim 1, wherein the base material is a formed body of a biocompatible substance.

3. The cell-supporting body according to claim 1, wherein the base material includes a formed body and a biocompatible substance applied to a surface of the formed body.

4. The cell-supporting body according to claim 1, wherein the biocompatible substance is a bio-derived polymer or a biodegradable synthetic polymer.

5. The cell-supporting body according to claim 4, wherein the biocompatible substance is at least one bio-derived polymer selected from the group consisting of collagen, gelatin, fibronectin, laminin, and a polysaccharide.

6. The cell-supporting body according to claim 4, wherein the biocompatible substance is at least one biodegradable synthetic polymer selected from the group consisting of polylactic acid, polyglycolic acid, poly(ε-caprolactone), polyvinyl alcohol, and a copolymer thereof.

7. The cell-supporting body according to claim 4, wherein the biocompatible substance is at least one biocompatible substance selected from the group consisting of collagen, gelatin, fibronectin, polyvinyl alcohol, and a copolymer thereof.

8. The cell-supporting body according to claim 1, wherein the biocompatible substance is a material selected according to an introduction rate of the gelatin particle to be achieved for a cell supported by the cell-supporting body.

9. The cell-supporting body according to claim 1, wherein the base material has a two-dimensional shape.

10. The cell-supporting body according to claim 1, wherein the base material has a three-dimensional shape.

11. The cell-supporting body according to claim 1, wherein the gelatin particle carries a reagent or an agent.

12. The cell-supporting body according to claim 11, wherein the reagent or the agent is a molecular beacon.

13. A manufacturing method of a cell-supporting body, comprising:
preparing a base material including a biocompatible substance; and
applying a gelatin particle to the base material to retain the gelatin particle on the base material.

14. The manufacturing method of a cell-supporting body according to claim 13, wherein the biocompatible substance is a material selected according to an introduction rate of the gelatin particle to be achieved for a cell supported by the cell-supporting body.

15. A cell culturing method, comprising:
preparing the cell-supporting body according to claim 1; and
seeding a cell on the cell-supporting body.

16. A cell structure, comprising:
the cell-supporting body according to claim 1 ; and
a cell retained on the cell-supporting body.

17. The cell-supporting body according to claim 2, wherein the base material includes a formed body and a biocompatible substance applied to a surface of the formed body.

18. The cell-supporting body according to claim 2, wherein the biocompatible substance is a bio-derived polymer or a biodegradable synthetic polymer.

19. The cell-supporting body according to claim 2, wherein the biocompatible substance is a material selected according to an introduction rate of the gelatin particle to be achieved for a cell supported by the cell-supporting body.

20. The cell-supporting body according to claim 2, wherein the base material has a two-dimensional shape.
